(19) United States
(12) Patent Application Publication
(10) Pub. No.: US 2003/0065297 A1
(43) Pub. Date: Apr. 3, 2003

(54) Process for manufacturing disposable fluid-handling article

(75) Inventors: James Harold Davis, Amelia, OH (US); Nezam Malakouti, Loveland, OH (US); Terrill Alan Young, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/252,175

(22) Filed: Sep. 23, 2002

Related U.S. Application Data
(60) Provisional application No. 60/325,847, filed on Sep. 28, 2001.

(51) Int. Cl. B29C 31/00; B32B 31/00; B29C 47/00; G05G 15/00; A61F 13/15; A61F 13/20

(52) U.S. Cl. 604/378; 604/384; 156/244.11; 156/229; 156/349

(57) ABSTRACT

A method and an apparatus for manufacturing disposable fluid-handling articles including absorbent articles (e.g., baby diapers, adult incontinence articles, feminine hygiene articles, baby swim diapers, dining bibs, wound dressing) and benefit-component-delivering articles (e.g., wash cloth, body wipes, body wraps, pet grooming articles, cleaning and polishing articles) are disclosed. The present invention can combine the web-forming technology with the web-converting technology into a continuous process for making a disposable fluid-handling article, wherein two or more of the components of the fluid-handling article are formed on the converting line from extruded polymeric materials. Thus, the new method and apparatus can reduce or eliminate the need for continuous webs of fabrics, films, foams, elastics, etc. that have been transported from web producers in a packaged form, e.g., wound rolls and festooned boxes.

This application claims the benefit of U.S. Provisional Application No. 60/325,847, filed Sep. 28, 2001.

### FIELD OF THE INVENTION

The present invention relates to the apparatus and methods suitable for manufacturing disposable fluid-handling articles including absorbent articles (e.g., baby diapers, adult incontinence articles, feminine hygiene articles, baby swim diapers, dining bibs, wound dressing) and benefit-component-delivering articles (e.g., wash cloth, body wipes, body wraps, pet grooming articles, cleaning and polishing articles).

### BACKGROUND

Disposable fluid-handling articles are often produced on high-speed converting lines using, for starting materials, continuous webs of fabrics, films, foams, elastics, etc. that have been transported from web producers in a packaged form (e.g., as wound rolls or festooned boxes), and are unpacked (e.g., unwound or de-festooned) in order to be fed as continuous webs into the converting line. In the converting lines, various converting operations work the webs to convert them into components of disposable fluid-handling articles that are eventually joined into a composite web that is finally cut into discrete final articles.

Unfortunately, packing and transporting continuous webs presents several problems. First, packing and transporting can often irreversibly change the web material, especially the webs that need to retain original, pre-packaged properties. For example, a soft, high-loft web can become continuously flat as a result of roll winding or intermittently deformed as a result of festooning. (When wound into a roll, the web is subjected to compression forces that are often needed for both retaining the web in the roll formation and for subsequent un-winding of the web from the roll. Also, when packaged in a festoon configuration into a box, the web often develops a permanent creep in the folded portions of the festooned web due to being bent and compressed.) Second, webs often need to be provided with special strength properties to make them suitable for roll winding or festooning. These properties often are achieved by applying to these webs special additives that can affect or compromise the desired properties of the final product and/or increase the cost of the web. Similar negative effects can take place when, prior to roll winding, the webs are sprayed with anti-static solutions to prevent or minimize in-layer subsequent sticking during un-winding of the web. Third, webs often require relatively expensive winding and un-winding high-speed automatic equipment and qualified personnel to operate and support it. Fourth, often the material properties that cannot be provided by a packaged web need to be provided by converting operations specially developed to make the web softer, thinner, thicker, elastic, absorbent, cloth-like, breathable, aesthetic, etc. These operations add more cost and time in developing new products.

Consequently, it would be beneficial to reduce or eliminate the need for packing and transporting the webs to the converting lines by providing a new process that is continuous from the material-forming steps to the converting steps. It would also be beneficial to provide a new process that opens up opportunities for producing new products that otherwise are prohibitively costly or not feasible with the packaged webs.

### SUMMARY OF THE INVENTION

In response to the difficulties and problems discussed above, a new process and apparatus for producing disposable fluid-handling articles have been discovered that can reduce or eliminate the need for packaged webs. The new process is a continuous process that links the steps of forming the webs with the steps of converting the webs into disposable fluid-handling articles.

In one aspect, the present invention concerns a method for manufacturing a disposable fluid-handling article that can comprise at least two primary components made continuously from bulk starting materials. The method comprises the steps of:

a) feeding a first polymeric bulk starting material into at least one first polymeric extrusion apparatus disposed adjacent to a first collecting surface moving at a first velocity in relation to the first polymeric extrusion apparatus;

b) extruding a first molten stream of a first polymeric material from the first polymeric extrusion apparatus;

c) continuously forming a first primary component of the disposable fluid-handling article from the first molten stream;

d) feeding a second polymeric bulk starting material into at least one second polymeric extrusion apparatus disposed adjacent to a second collecting surface moving at a second velocity in relation to the second polymeric extrusion apparatus;

e) extruding a second molten stream of a second polymeric material from the second polymeric extrusion apparatus;

f) continuously forming a second primary component of the disposable fluid-handling article from the second molten stream;

g) joining the first and second primary components into a composite web, wherein the first primary component overlaps at least partially with the second primary component; and

h) severing the composite web in a direction generally perpendicular to a machine direction so as to form the disposable fluid-handling article.

In another aspect, the present invention concerns an apparatus, which is a production line for producing a disposable fluid-handling article that can comprise at least two primary components made continuously from bulk starting materials. The production line comprising:

(a) a first primary component station for providing a first primary component, wherein the first primary component station includes at least one first extrusion module for forming the first primary component from one or more first polymeric bulk starting material by extrusion and formation of the first primary component continuously on the production line; and

(b) a second primary component station adjacent to the first primary component station for providing a second primary component, wherein the second primary component station includes at least one second extrusion module for forming the second primary component from one or more second polymeric bulk starting material by extrusion and formation of the second primary component continuously on the production line.

### BRIEF DESCRIPTION SHOWN IN THE DRAWINGS

While the specification concludes with claims particularly pointing out and distinctly claiming the subject matter which is regarded as the present invention, it is believed that the invention will be more fully understood from the following description taken in conjunction with the accompanying drawings, in which:

FIG. 1 is a plan view of an exemplary diaper that can be produced by the method and apparatus of the present invention; the diaper is shown in a flat-out state, wherein the wearer-facing side of the diaper is oriented towards the viewer and portions of the diaper structure are cut-away to more clearly show the construction of the diaper;

FIG. 2 is a cross-sectional view of the diaper in FIG. 1 taken along the cut line 2-2;

FIG. 3 is a is a simplified elevational view, in the form of a block diagram, of one embodiment of a disposable fluid-handling article production line of the present invention capable of producing the exemplary diaper shown in FIGS. 1-2;

FIG. 4 is a simplified elevational view of a spun-bonded module;

FIG. 5 is a simplified elevational view of a melt-blown module;

FIG. 6 is a simplified elevational view of a film-forming module;

FIG. 7 is a simplified elevational view of one embodiment of the backsheet station of the present invention;

FIG. 8 is a simplified elevational view of another embodiment of the backsheet station of the present invention;

FIG. 9 is a simplified elevational view of another embodiment of the backsheet station of the present invention;

FIG. 10 is a simplified elevational view of another embodiment of the backsheet station of the present invention;

FIG. 11 is a simplified elevational view of another embodiment of the backsheet station of the present invention;

FIG. 12 is a simplified elevational view of another embodiment of the backsheet station of the present invention;

FIG. 13 is a simplified elevational view of another embodiment of the backsheet station of the present invention;

FIG. 14 is a simplified elevational view of another embodiment of the backsheet station of the present invention;

FIG. 15 is a simplified elevational view of one embodiment of the core station of the present invention;

FIG. 16 is a simplified elevational view of another embodiment of the core station of the present invention;

FIG. 17 is a simplified elevational view of another embodiment of the core station of the present invention;

FIG. 18 is a simplified elevational view of another embodiment of the core station of the present invention;

FIG. 19 is a simplified elevational view of another embodiment of the core station of the present invention;

FIG. 20 is a simplified elevational view of one embodiment of the topsheet station of the present invention;

FIG. 21 is a simplified elevational view of another embodiment of the topsheet station of the present invention;

FIG. 22 is a simplified elevational view of another embodiment of the topsheet station of the present invention;

FIG. 23 is a simplified elevational view of another embodiment of the topsheet station of the present invention;

FIG. 24 is a simplified elevational view of another embodiment of the topsheet station of the present invention;

FIG. 25 is a simplified elevational view of one embodiment of the leg cuff station of the present invention;

FIG. 26 is a simplified elevational view of another embodiment of the leg cuff station of the present invention; and

FIG. 27 is a simplified elevational view of another embodiment of the leg cuff station of the present invention.

### DETAILED DESCRIPTION OF THE INVENTION

The method and the apparatus of the present invention can reduce or eliminate the need for packing and transporting the webs from a web-producing facility to a web converting facility producing disposable fluid-handling articles. The present invention can combine the web-forming technology with the web-converting technology into a continuous process for making a disposable fluid-handling article, wherein two or more of the components of the fluid-handling article are formed on the converting line from extruded polymeric materials.

Terminology

A “web-producing facility” refers herein to a production facility producing continuous webs that are packaged for transportation as wound rolls or festooned containers.

A “converting facility” refers herein to any production facility producing a finished disposable fluid-handling article that is complete for use by a consumer, or one or more components of a disposable fluid-handling article intended for assembling into a finished disposable fluid-handling article.

The term “disposable fluid-handling articles” refers herein to both disposable absorbent articles and disposable benefit-component-delivering articles.

A “disposable absorbent article” refers herein to a device that normally absorbs and retains fluids. In certain instances, the phrase refers to devices that are placed against or in proximity to the body of the wearer to absorb and contain the excreta and/or exudates discharged from the body, and includes such personal care articles as baby diapers, baby training pants, adult incontinence articles, feminine hygiene articles, baby swim diapers, wound dressing, and the like. In other instances, the phrase refers to protective articles, such as, for example, dining bibs that have the ability to absorb food items to prevent staining of the wearer's clothing.

A “disposable benefit-component-delivering article” refers herein to a device that can retain a benefit component until such time when the article is utilized by a consumer for its intended purpose. The benefit component can include, for example, a lotion, a shampoo, a soap, a polishing material, a cleansing material, or the like, and such devices can include wash cloth, body wipes, body wraps, pet grooming articles, cleaning and polishing articles, and the like.

The term “disposable” is used herein to describe products which generally are not intended to be laundered or otherwise restored or extensively reused in their original function, i.e., preferably they are intended to be discarded after about 10 uses or after about 5 uses or after about a single use. It is preferred that such disposable articles be recycled, composted or otherwise disposed of in an environmentally compatible manner.

The term “diaper” includes baby diapers, baby training pants, baby pool diapers, or adult incontinence articles and refers to a disposable fluid-handling article generally worn by infants and other incontinent persons about the lower torso.

The term “feminine hygiene articles” refers herein to any fluid-handling article worn by women to absorb and contain menses and other vaginal exudates.

A “body wrap” refers herein to an article or a garment worn about the body, typically to provide some therapeutic benefit, such as, for example, pain relief, wound coverage or to hold another device or article near the body.

The term “web” is meant herein any continuous material, including a film, a nonwoven fabric, a foam or a combination thereof, or a dry lap material including wood pulp, and the like, having a single layer or multiple layers.

The term “non-woven fabric” or “non-woven material” or “non-woven web” or “non-woven” refers herein to a material made from continuous filaments and/or discontinuous fibers, without weaving or knitting by processes such as spun-bonding and melt-blowing. The non-woven material can comprise one or more layers of the nonwoven material, wherein each layer can include continuous filaments or discontinuous fibers.

The term “foam” refers herein to any material comprising a solid, liquid crystalline, or liquid continuous phase and a gaseous dispersed phase. Because of the dispersed gaseous phase, a foam has a density less than the density of the continuous phase.

The term “film” refers herein to any polymeric film made by a process that includes extrusion of a polymeric material through a narrow slot of a die. The polymeric film can be impervious to a liquid and pervious to an air vapor.

An “elastomer” refers herein to a polymer exhibiting elastic properties.

The term “elastic” refers herein to any material that upon application of a force to its relaxed, initial length can stretch or elongate to its elongated length without rupture and breakage, and which can substantially recover its initial length upon release of the applied force.

The term “polymer” or “polymeric” refers herein to thermosetting and thermoplastic polymers and polymeric compositions, including but not limiting to polyolefins (such as polyethylene and polypropylene), polyesters, polyamides, polyurethanes, superabsorbent materials, rayon, Kevlar, and blends and copolymers, biconstituent or bicomponent mixtures thereof and the like. The polymeric material can also include various pigments to provide desired colors and/or visual effects.

A “natural material” means herein that a material derived from plants, animals, insects or byproducts of plants, animals, and insects. Non-limiting examples of natural materials useful in the disposable articles include cellulosic fibers, cotton fibers, keratin fibers, silk fibers and the like. Non-limiting examples of cellulosic fibers include wood pulp fibers, hemp fibers, jute fibers, and the like. Non-limiting examples of keratin fibers include wool fibers, camel hair fibers, and the like.

A “polymeric extrusion apparatus” refers herein to any machine capable of extruding a molten stream of a polymeric material through one or more orifices or slots of a die.

The term “extrude” or “extruding” refers herein to a process by which a heated polymer is forced through one or more orifices or slots of a die to form a molten stream.

The term “forming a component of a disposable absorbent article” refers herein to a continuous process wherein a primary component or an auxiliary component of a disposable fluid-handling article is produced continuously from a polymeric bulk starting material by extruding and forming one or more layers of the polymeric material.

The term “continuously form a component of a disposable fluid-handling article” refers herein to a continuous process wherein a primary component and/or an auxiliary component of a disposable fluid-handling article is created continuously from a molten stream of a polymeric material that has been fed in the form of a polymeric bulk starting material into a polymeric extruding apparatus for extruding as the molten stream.

The term “continuous process” refers herein to a process wherein at least one molten stream of a polymeric material is substantially formed into a primary and/or an auxiliary component of a disposable fluid-handling article, and wherein this at least one molten stream is not formed into any packaged web, e.g., a wound roll or a festooned box.

The term “polymeric bulk starting material” refers herein to any material, suitable for use in production of a disposable fluid-handling article or a component of a disposable fluid-handling article, that is polymeric and is provided in bulk form including solids, semisolids, or solutions of one or more polymeric materials. In the solid form, the polymeric bulk starting materials can be supplied as pellets, granules, or particles.

The term “bulk starting materials” refers herein to polymeric bulk starting materials and non-polymeric bulk starting materials such as wood pulp, natural fibers, and the like.

A “spun-bonded apparatus” refers herein to a machine capable of producing a molten stream of a polymeric material in a form of continuous filaments.

The term “continuous filaments” refers herein to substantially endless strings of a polymeric material extruded through a multiplicity of small orifices of a die.

The term “melt-blown apparatus” refers herein to a machine capable of producing a molten stream of a polymeric material in a form of discontinuous fibers.

The term “discontinuous fibers” or “melt-blown fibers” refers herein to limited-length strings of a polymeric material, which are normally produced by fragmenting one or more continuous filaments by a stream of hot gas (typically air) and having a length ranging from about 5 mm to about 500 mm and a diameter less than about 20 microns.

The term “film forming apparatus” refers herein to a machine capable of producing a molten stream of a polymeric material in a form of a film.

The term “molten stream” refers herein to one or more streams of continuous filaments, discontinuous fibers, or continuous films of a polymeric material exiting a polymeric extrusion apparatus for forming the molten stream into a component of a disposable fluid-handling article. It should be noted that the term “molten stream” excludes herein any stream of any melt adhesive typically used for the purpose of adhesively bonding the layers or components of a disposable fluid-handling article.

The term “joined” herein encompasses configurations whereby a component of a disposable fluid-handling article is secured directly or indirectly (by one or more intermediate members) to another component of the disposable fluid-handling article. The securing means can include any means known in the art, for example, adhesives, heat bonds, pressure bonds, ultrasonic bonds, and the like.

Exemplary Fluid-Handling Article

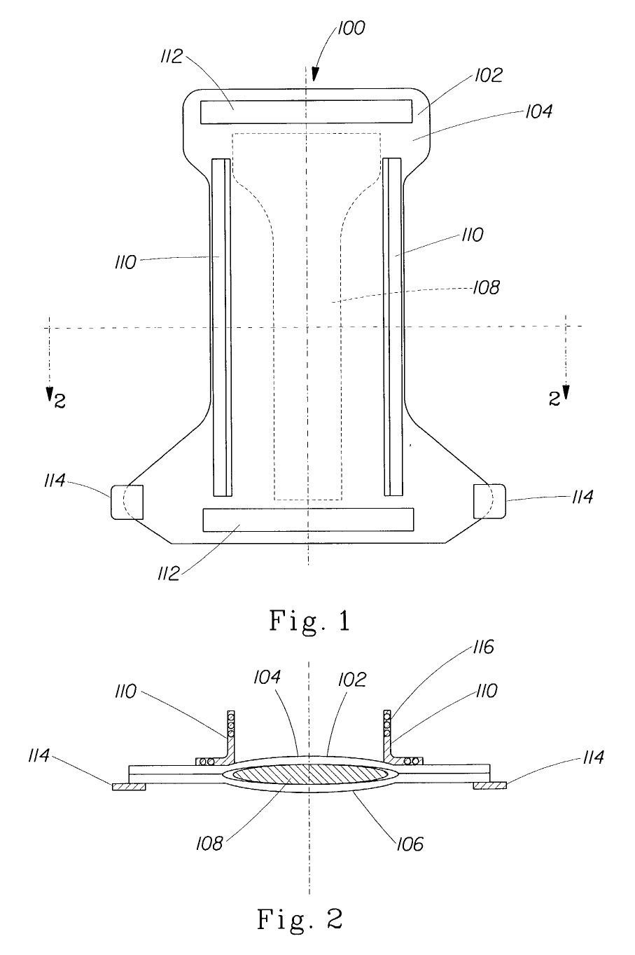

One example of a disposable fluid-handling article that can be produced by the process and the apparatus of the present invention is illustrated in FIGS. 1 and 2. In FIG. 1, the diaper 100 is shown in a plan view, in a flat-out state, wherein the wearer facing side 102 of the diaper 100 is oriented towards the viewer. FIG. 2 shows a cross-sectional view of the laminate construction of the diaper 100.

The laminate construction of the diaper 100 includes three primary components: a liquid-permeable topsheet 104, a liquid-impervious backsheet 106 opposing the topsheet 104, and an absorbent core 108 positioned between at least a portion of the topsheet 104 and the backsheet 106. These primary components provide primary functions of an absorbent article of absorbing and retaining the fluid, and also normally define the size, the shape, and the perimeter of a particular disposable article. The diaper 100 further includes three auxiliary components: leg cuffs 110 having strands of elastic material 116, a waist feature 112, and fasteners 114. The auxiliary components normally provide functions complementing the function of the primary components, for example, leg cuffs provide improved leakage protection around the wearer's legs, waist bands provide improved fit of the diaper around the wearer, and fasteners hold the diaper around the wearer. It should be noted that the number of components, which include primary and auxiliary components, could vary depending on a particular product design.

Production Line

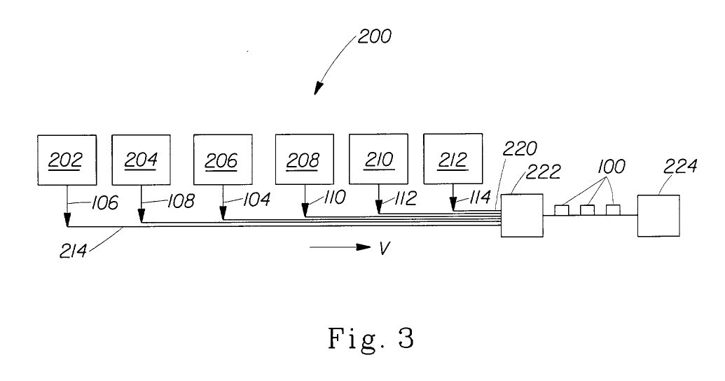

FIG. 3 is a simplified elevation view, in the form of a block diagram, of one embodiment of a production line 200 of the present invention for producing disposable fluid-handling articles, in particular, the diaper 100 shown in FIGS. 1 and 2. The production line 200, depending on the complexity of the disposable liquid-handling article being produced, can include any number of stations, wherein each station can provide a particular component of the disposable fluid-handling article for joining with the rest of the components into a final product. In addition, the sequential order of the stations can vary depending upon the type of a disposable fluid-handling article being produced, its particular design, as well as many other process and production considerations. Further, the production line 200 can be configured to form any suitable configuration of the stations on the production floor. For example, in FIG. 1, the production line 200 is shown to have a rectilinear configuration of the stations, however, the configuration can be curvilinear, circular, U-shape, C-shape, X-shape, cross-shape or any combination thereof. Further, several production lines 200 can be situated in any suitable relation to each other to form any suitable arrangement on the production floor.

For producing the exemplary diaper 100 having six components, the production line 200 can include six component stations that include three primary component stations and three auxiliary component stations. The primary component stations include a backsheet station 202, a core station 204, and a topsheet station 206 for providing, respectively, three primary components of the diaper 100: the backsheet 106, the core 108, and the topsheet 104. The auxiliary component stations can include a leg cuff station 208, a waist feature station 210, and a fastener station 212 for providing, respectively, the three auxiliary components of the diaper 140: the leg cuff 110, the waist feature 112, and the fastener 114.

As shown in FIG. 3, each of the provided components is deposited onto a moving surface. The backsheet 106 is deposited onto a moving surface 214, which can be a conveying surface, and the rest of the components are deposited on top of each other and joined to form a composite web 220. The composite web 220 is then cut by a final knife 222 into individual diapers 100. The diapers 100 can then be packaged at a packaging operation 224 into any suitable size and shape packages containing any suitable number of diapers.

Any of the primary component stations 202, 204, and 206 or any of the auxiliary component stations 208, 210, and 212 of the production line 200 can include one or more extrusion apparatus that can extrude and continuously form a component of a disposable fluid-handling article. An extrusion apparatus can include a spun-bonded module for providing continuous filaments, and/or a melt-blown module for providing discontinuous fibers, and/or a film-forming module for providing a continuous film.

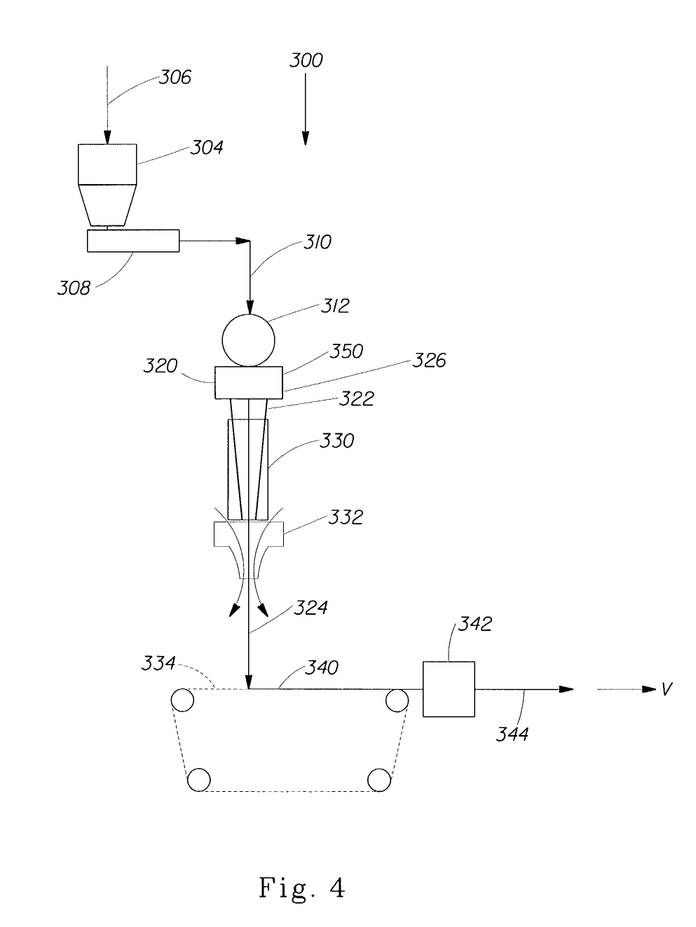

FIGS. 4, 5, and 6 show, respectively, a spun-bonded module 300, a melt-blown module 400, and a film module 500. Each of the extrusion modules 300, 400, and 500 can include a hopper 304 for collecting a polymeric bulk starting material 306, an extruder 308 for melting the polymeric bulk starting material 306 into a melt 310, and a pump 312 for creating a uniform flow of the melt 310. (It should be noted that the extrusion modules 300, 400, and 500 do not need to include the hopper 304 when the polymeric bulk starting material 306 can be supplied to the extruder 308 by any suitable conventional material feeding system including but not limited to a chute or a pipe.)

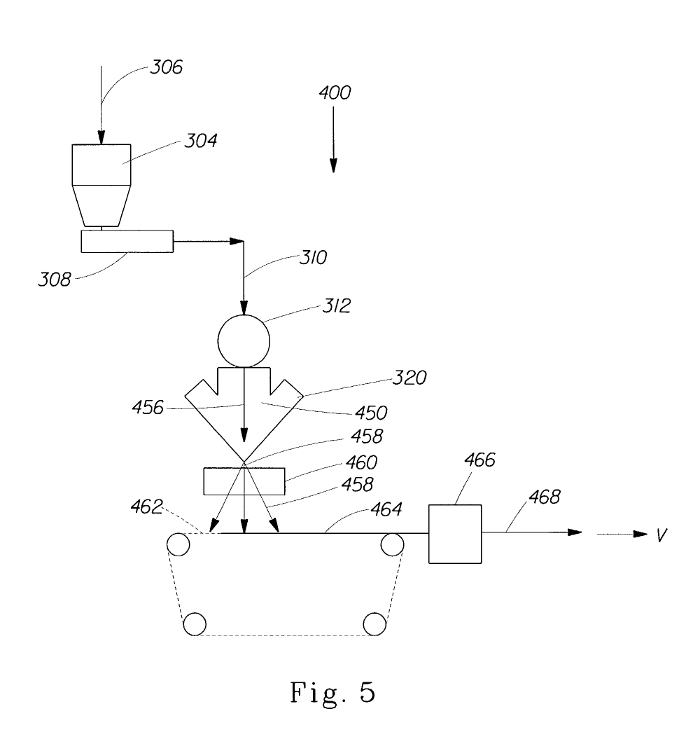

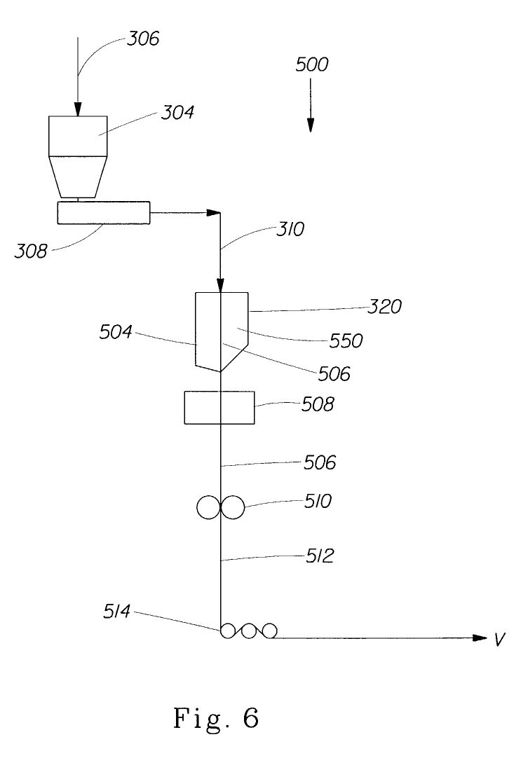

Each of the extrusion modules 300, 400, and 500 can further include a polymeric extrusion apparatus 320 for extruding the melt 310 as a molten stream. The polymeric extrusion apparatus 320 can be a spun-bonded apparatus 350 (see FIG. 4), a melt-blown apparatus 450 (see FIG. 5), or a film-forming apparatus 550 (see FIG. 6).

Referring to FIG. 4, the spun-bonded module 300 extrudes a molten stream 322 in the form of continuous filaments 324 through a die 326 of the spun-bonded apparatus 350. The spun-bonded module 300 can further include a cooling device 330 for cooling the continuous filaments 324, a drawing device 332 for stretching the continuous filaments 324 to reduce their cross-sectional diameter, a moving collecting surface 334 for depositing the continuous filaments 324 to form a layer 340 of continuous filaments 324, and a bonding apparatus 342 for creating bonds between continuous filaments 324 to form a coherent layer 344 of continuous filaments 324 by thermal bonding, adhesive bonding, or by entanglement of the continuous filaments.

Bonding of continuous filaments 324 can be facilitated by application of pressure or heat and pressure, if the filaments are not hot enough, to form thermal fusion or adhesive bond between adjacent filaments. The pressure applicators can include contacting or non-contacting means to bring adjacent filaments in intimate contact. Contacting means can include heated or non-heated compacting or calendaring rolls having smooth or textured surface(s). Non-contacting means can include various pressure differential techniques, including compressed gas and/or vacuum. The continuous filaments 324 can be laid down on the collecting surface 334 in a desired orientation by one or more of the following methods including but not limited to by rotating the extrusion die 326, by electrical charges, by controlled fluid streams, and by the travel velocity V1 of the collecting surface 334 collecting the continuous filaments 324.

The collecting surface 334 can have openings for the blown gas to escape at least partially therethrough with or without assistance of a vacuum. The collecting surface 334 can include a screen, a perforated belt, a woven belt, a non-woven belt, a layer of spun-bonded filaments, a layer of melt-blown filaments, a porous film, or any combination thereof. The side of the collecting surface that faces the molten stream of the polymeric material can have any suitable shape, for example, flat, round, concave, convex. This side can have protrusions or projections, cavities or depressions, or any combination thereof. The openings of the collecting surface can be of any size and shape to provide an open area, which is suitable for the blown gas to escape at least partially therethrough, and for the filaments not to exit therethrough.

The spun-bonded module 300 can be manufactured using any suitable commercial hardware normally used in production of non-woven materials and produced by such suppliers as, for example, Asson Engineering Inc., Fla. 33301; Hills, Florida 32904; Reifenhauser, Germany; JM Laboratories of Nordson, Ga., 30534; and Kobelco, Japan.

Referring to FIG. 5, the polymeric extrusion apparatus 320 is the melt-blown apparatus 450 of the melt-blown module 400. The melt-blown apparatus 450 can extrude a molten stream 456 in the form of discontinuous fibers 458 by fragmenting the melt by a pressurized hot gas (typically, air).

The melt-blown module 400 can also include a cooling device 460 for cooling the discontinuous melt-blown fibers 458 before depositing the cooled fibers 458 onto a moving collecting surface 462 to form a layer 464 of melt-blown fibers 458. The collecting surface 462 of the melt-blown module 400 can be similar in all or any aspects to the collecting surface 334 of the spun-bonded module 300 described in detail hereinabove.

The melt-blown module 400 can further include a bonding apparatus 466 for creating bonds between the melt-blown fibers 458 to form a coherent layer 468 by thermal bonding, adhesive bonding, or entanglement of the melt-blown fibers 258. The bonding apparatus 466 of the melt-blown module 400 can be similar in all or any aspects to the bonding apparatus 342 of the spun-bonded module 300 described in detail hereinabove.

The melt-blown module 400 can be manufactured using any suitable commercial hardware normally used in production of non-woven materials and produced by such suppliers as, for example, Asson Engineering Inc., Florida 33301; Hills, Fla. 32904; Reifenhauser, Germany; JM Laboratories of Nordson, Ga., 30534; and Kobelco, Japan.

Referring to FIG. 6, the polymeric extrusion apparatus 320 is the film-extruding apparatus 550 of the film-forming module 500. The film-extruding apparatus 550 can include an extrusion die 504 having a slot for extruding a molten stream 506 in the form of a film. The film-forming module 500 can further include a cooling device 508 for cooling the molten stream 506, a casting device 510 for forming the cooled molten stream 506 into a desired thickness film 512 before depositing the film 512 onto a moving collecting surface 514. The collecting surface 514 of the film-forming module 500 can be similar in all or any aspects to the collecting surface 334 of the spun-bonded module 300 described in detail hereinabove. The film-forming module 500 can be manufactured using any suitable commercial hardware normally used in production of non-woven materials and produced by such suppliers as, for example, Asson Engineering Inc., Florida 33301; Hills, Fla. 32904; Reifenhauser, Germany; JM Laboratories of Nordson, Ga., 30534; and Kobelco, Japan.

Backsheet Station

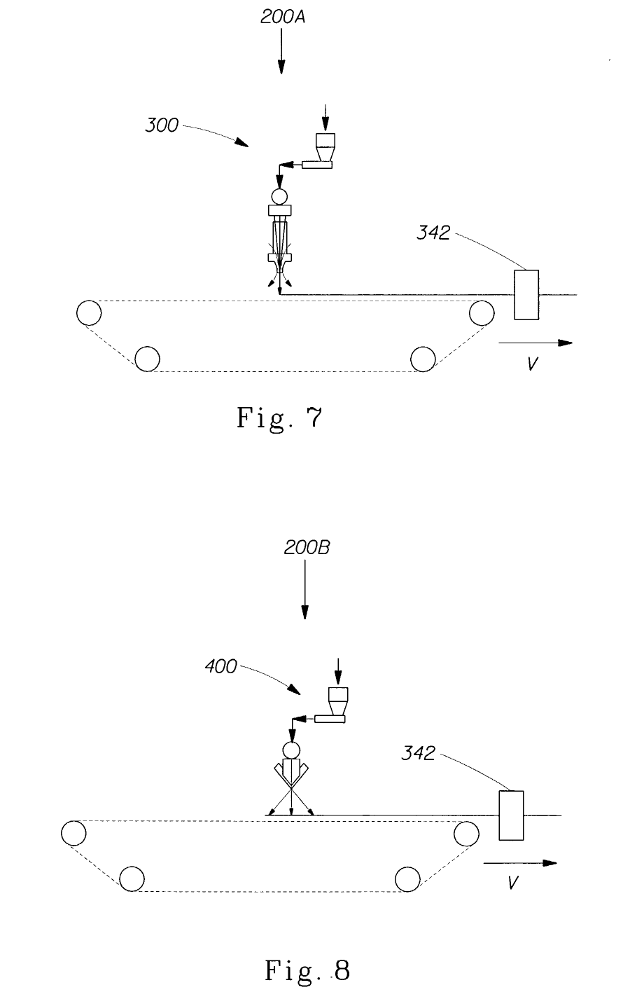

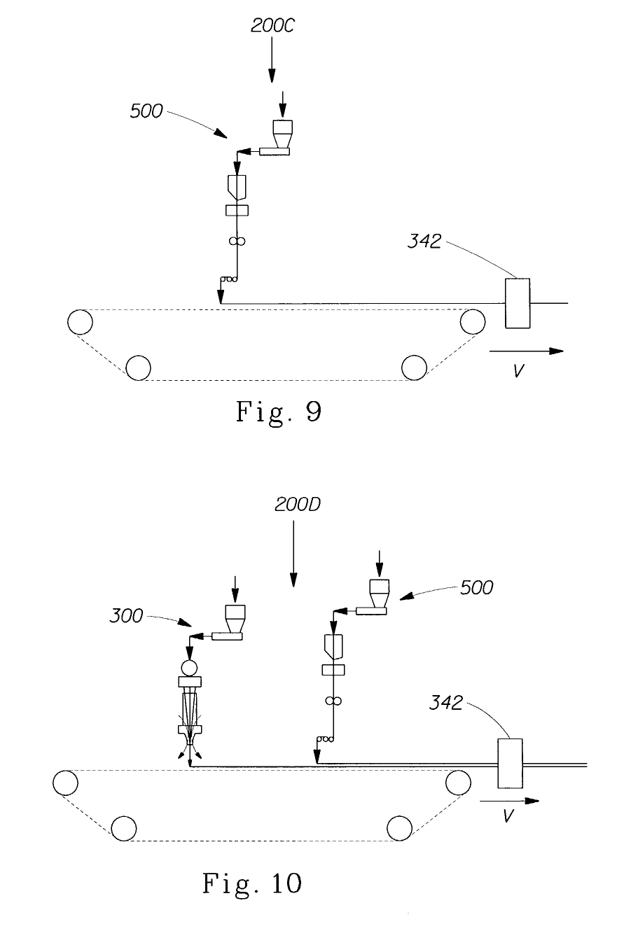

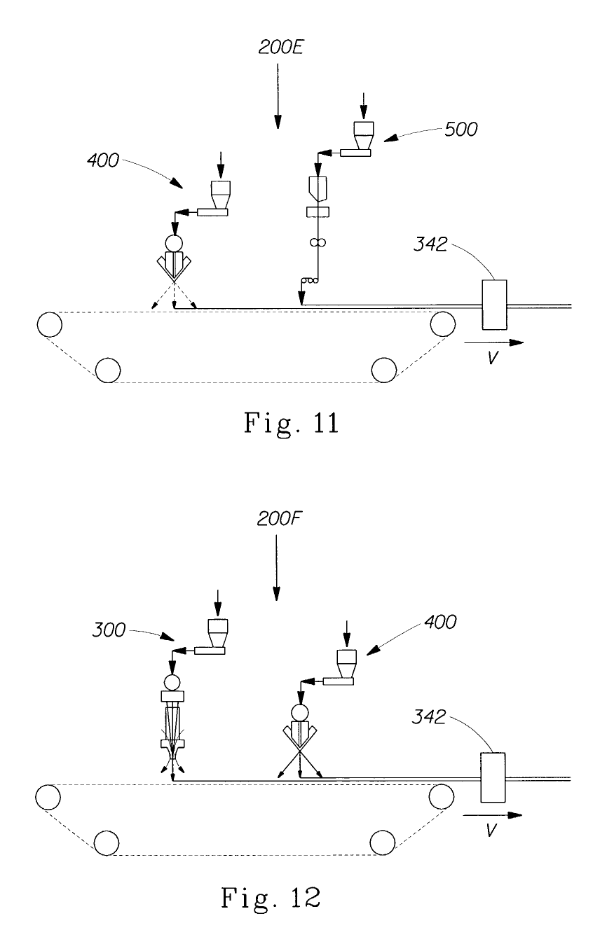

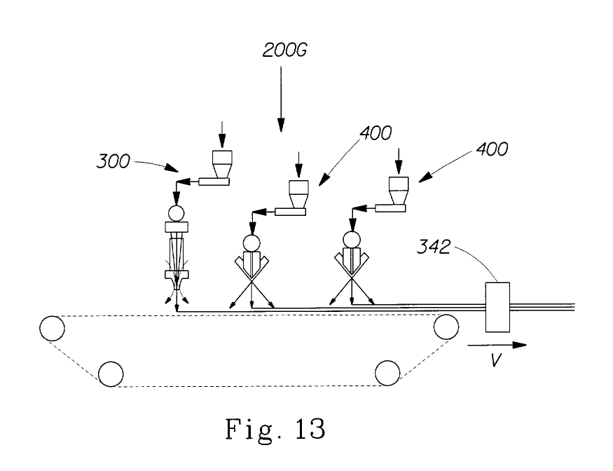

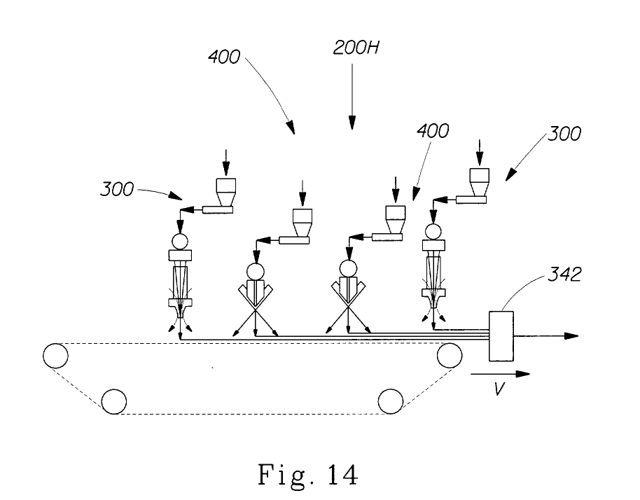

The backsheet station 202 of the production line 200 shown in FIG. 3 can include one or more spun-bonded module 300 (see FIG. 4), and/or one or more melt-blown module 400 (see FIG. 5), and/or one or more film-forming module 500 (see FIF. 6). The modules 300, 400, and 500 can be situated on the production line 200 in any suitable combination. FIGS. 7-14 illustrate exemplary, non-limiting embodiments of the backsheet station 202 for producing the backsheet 106 of the diaper 100 shown in FIGS. 1-2. FIG. 7 shows a backsheet station 202A having one spun-bonded module 300; FIG. 8 shows a backsheet station 202B having one melt-blown module 400; FIG. 9 shows a backsheet station 202C having one film-forming module 500; FIG. 10 shows a backsheet-station 200D having one spun-bonded module 300 and one film-forming module 500; FIG. 11 shows a backsheet station 200E having one melt-blown module 400 and one film-forming module 500; FIG. 12 shows a backsheet station 200F having one spun-bonded module 300 and one melt-blown module 400; FIG. 13 shows a backsheet station 200G having one spun-boded module 300 and two melt-blown modules 400; and FIG. 14 shows a backsheet station 200H having two spun-bonded modules 300 and two melt-blown modules 400.

In other instances, the backsheet 106 can be provided by a backsheet-feeding module that can feed a suitable backsheet material supplied to the production line 200 in any suitable packaged form, for example, wound rolls or festoon containers. The topsheet-feeding module can utilize any suitable hardware commonly used in converting operations for feeding continuous forms of materials.

Core Station

The core station 204 of the production line 200 shown in FIG. 3 can include one or more spun-bonded module 300 (see FIG. 4), and/or one or more melt-blown module 400 (see FIG. 5), and/or one or more film-forming module 500 (see FIG. 6). The modules 300, 400, and 500 can be situated on the production line 200 in any suitable combination. The modules 300, 400, and 500 can use any suitable non-absorbent and/or absorbent (or superabsorbent) polymeric material that can be extruded and formed into one or more layers of continuous filaments and/or discontinuous fibers.

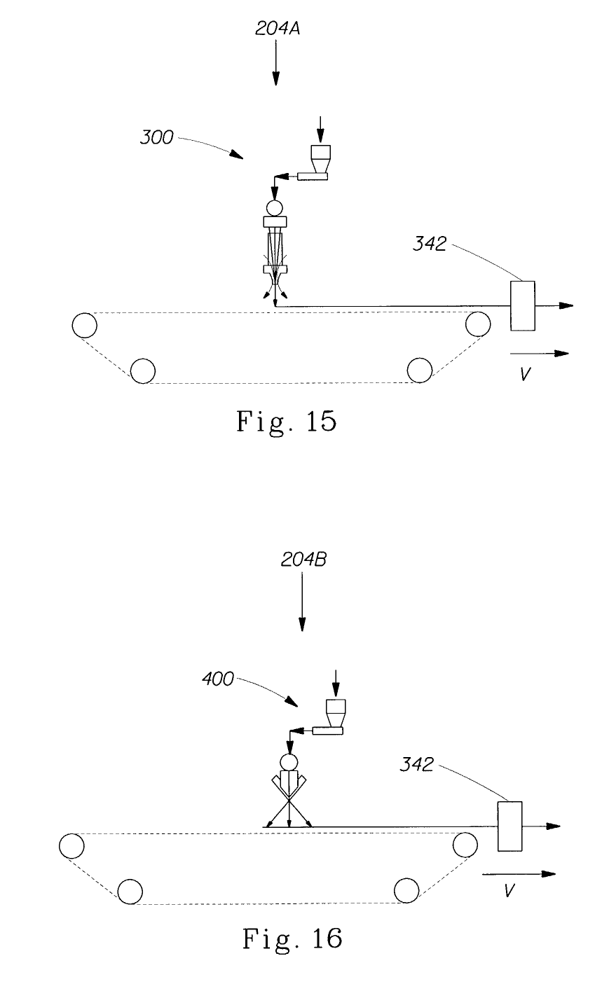

FIG. 15 shows a core module 204A having one spun-bonded module 300 providing continuous filaments of an absorbent polymeric material. Similarly, FIG. 16 shows a core module 204B having one melt-blown module 400 to provide discontinuous fibers of an absorbent polymeric material.

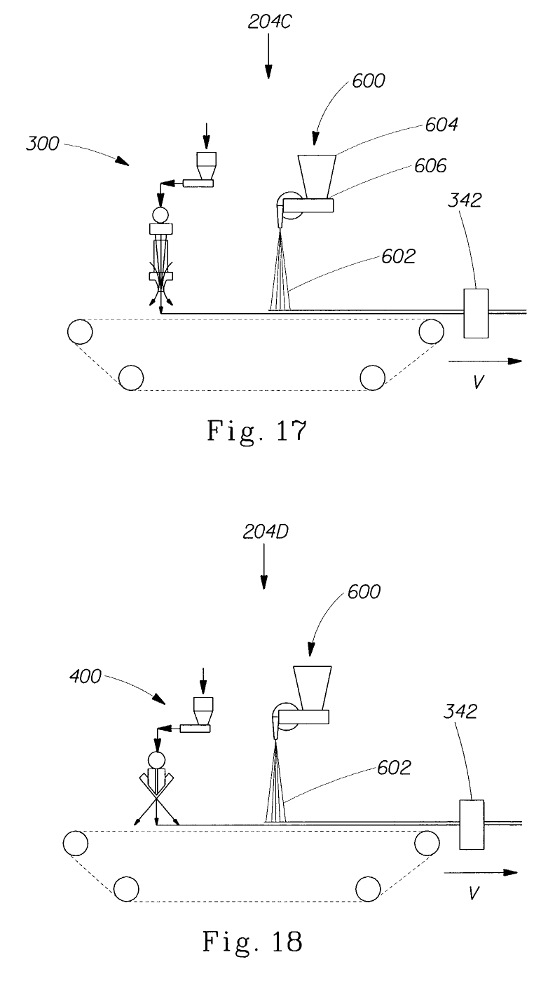

The core station 204 can also include one or more particle-feeding module 600 for dispensing superabsorbent particles 602, as shown, for example, in FIGS. 17-19, to form a core 108 of the diaper 100, including superabsorbent particles. The particle-feeding module 600 can include a hopper 604 for collecting the superabsorbent particles 602, a metering device 606 for creating a uniform flow of the particles 602. (However, it should be noted that the particle-feeding module 600 does not need to include the hopper 604 when the particles 604 can be supplied to the metering device 606 by any suitable material feeding system including but limited to a chute or a pipe.) The particle-feeding module 600 can be manufactured using any suitable commercial hardware, for example, the hopper 604 and the feeding device 606 are common products of the Acrison Corporation.

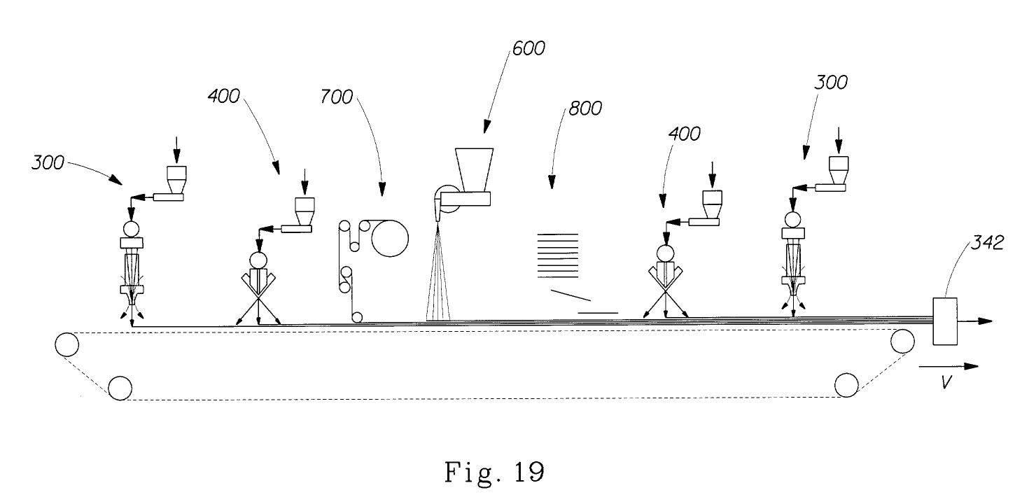

The core station 204 can also include a web-feeding module 700, as shown, for example, in FIG. 19, for providing any suitable web material that has been supplied in a packaged form (e.g., a roll or a festooned box) to the production line 200 to be used in certain embodiments of the core 108 of the diaper 100. Further, the core station 204 can include a sheet-feeding module 800 for providing any suitable discrete sheets that have been supplied in any suitable packaged form to the production line 200 for use in certain embodiments of the core 108 of the diaper 100.

In other instances, the core 108 of the diaper 100 can be provided by a core-feeding module that can feed the core 108 supplied to the production line 200 pre-fabricated and packaged as discrete cores or as a continuous web including the cores and packaged as, for example, wound rolls or festoon containers. The core-feeding module can utilize any suitable hardware commonly used in converting operations for feeding discrete or continuous forms of materials.

Topsheet Station

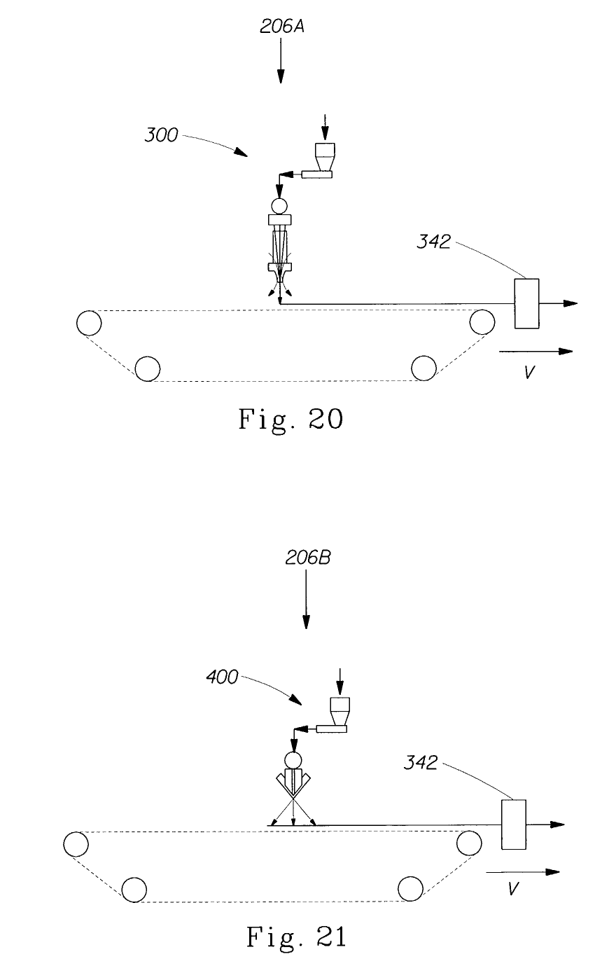

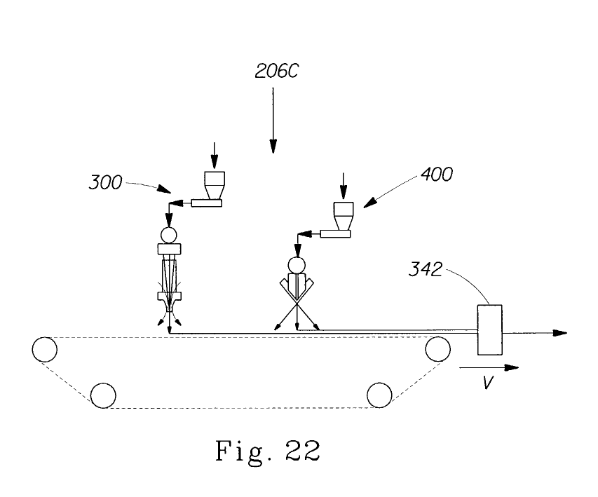

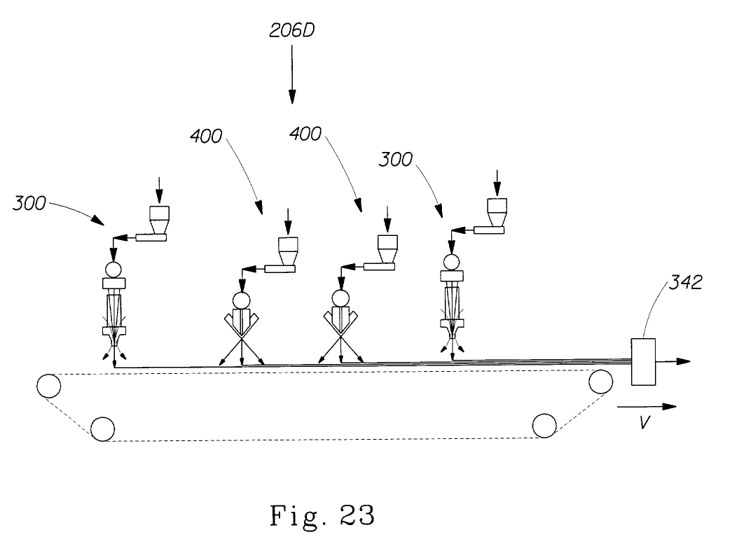

The topsheet station 206 of the production line 200 shown in FIG. 3 can include one or more spun-bonded module 300 (see FIG. 4), and/or one or more melt-blown module 400 (see FIG. 5), and/or one or more film-forming module 500 (see FIG. 6). FIGS. 20-23 show exemplary, non-limiting embodiments of the topsheet station 206 for producing the topsheet 104 of the diaper 100 shown in FIGS. 1-2. FIG. 20 shows a topsheet station 206A having one spun-bonded module 300; FIG. 21 shows a topsheet station 206A having one melt-blown module 400; FIG. 22 shows a topsheet station 206C having one spun-bonded module 300 and one melt-blown module 400; and FIG. 23 shows a topsheet station 206D having two spun-bonded modules 300 and two melt-blown modules 400.

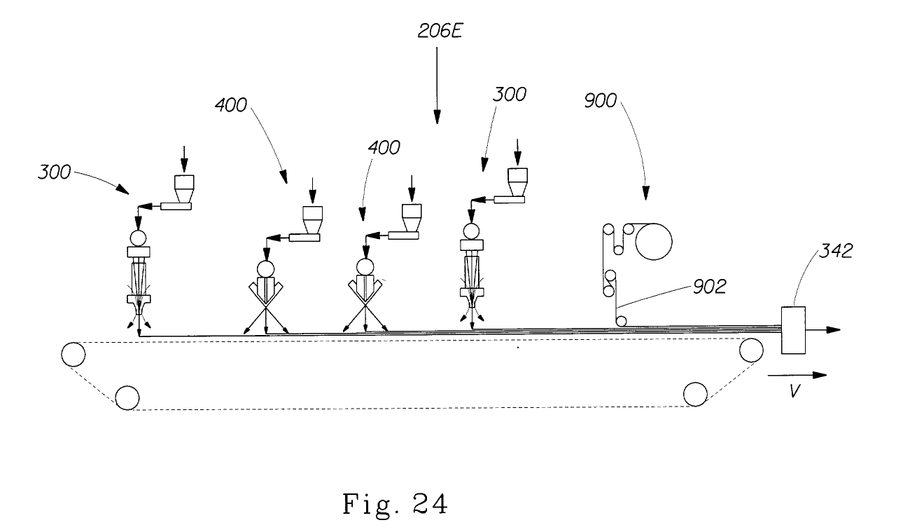

Optionally, the topsheet station 206 can also include one or more elastomer-feeding module 900 for feeding one or more of continuous strands of elastic material 902 as shown, for example, in FIG. 24, to form, if desired, an elasticized topsheet including elastic strands. The elastomer-feeding module 900 can utilize any suitable hardware commonly used in converting operations for feeding elastic strands. The modules 300, 400, 500, and 900 can be situated on the production line 200 in any suitable combination

In other instances, the topsheet 104 can be provided by a topsheet-feeding module that can feed a suitable topsheet material supplied to the production line 200 in any continuous form, such as, for example, wound rolls or festoon containers. The topsheet-feeding module can utilize any suitable hardware commonly used in converting operations for feeding continuous forms of materials.

Leg Cuff Station

The leg cuff station 208 of the production line 200 shown in FIG. 3 can include one or more spun-bonded module 300 (see FIG. 4), and/or one or more melt-blown module 400 (see FIG. 5), and/or one or more film-forming module 500. The leg cuff station 208 can also include one or more elastomer-feeding module 900, described above. The modules 300, 400, 500, and 900 can be situated on the production line 200 in any suitable combination.

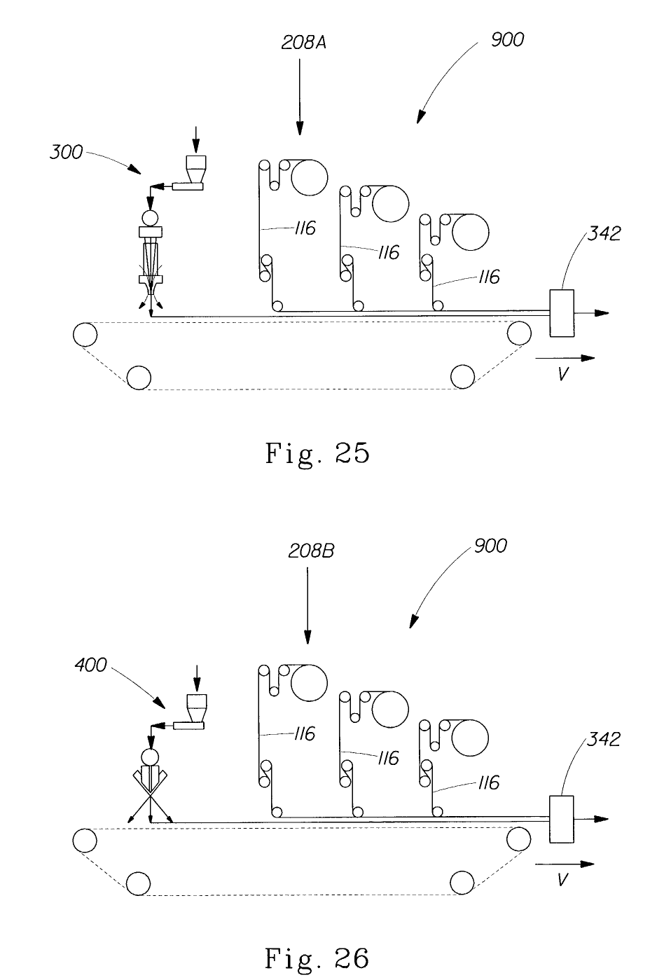

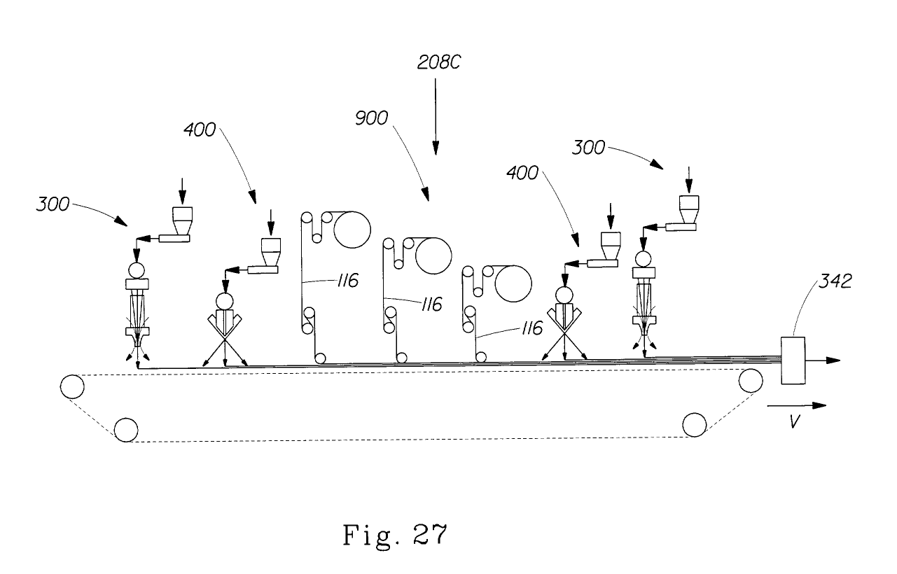

FIGS. 25-27 show exemplary, non-limiting embodiments of the leg cuff station 208 for producing the leg cuff 110 with elastics 116 of the diaper 100 shown in FIGS. 1-2. FIG. 25 shows a leg cuff station 208A having one spun-bonded module 300 and one elastomer-feeding module 900 feeding continuous strands of elastic materials 116; FIG. 26 shows a leg cuff station 208B having one melt-blown module 400 and one elastomer-feeding module 900; and FIG. 27 shows a leg elastic module 208C having two spunbonded modules 300, two melt-blown modules 400, and one elastomer-feeding module 900.

In other instances, the leg cuff 110 can be provided by a leg cuff-feeding module that can feed the leg cuff 110 supplied to the production line 200 pre-fabricated in any continuous form, such as, for example, wound rolls or festoon containers. The leg cuff-feeding module can utilize any suitable hardware commonly used in converting operations for feeding continuous forms of materials.

Waist Feature Station

The waist feature 112 of the diaper 100 of FIG. 1 and 2 can be provided by the waist module 210 that can include any combination of spun-bonded modules 300 (see FIG. 4) and/or melt-blown modules 400 (see FIG. 5) and/or film-forming modules 500 (see FIG. 6) and/or elastomer-feeding modules 900. In other instances, the waist feature 112 can be provided by a waist-feeding module that can feed the waist feature 112 supplied to the production line 200 in any continuous form, such as, for example, wound rolls or festoon containers. The waist feature-feeding module can utilize any suitable hardware commonly used in converting operations for feeding continuous forms of materials.

Fastener Station

The fastener 114 of the diaper 100 of FIGS. 1 and 2 can be provided by the fastener module 212 that can include any combination of spun-bonded modules 300 (see FIG. 4) and/or melt-blown modules 400 (see FIG. 5) and/or film-forming modules 500 (see FIG. 6). In other instances, the fastener 114 can be provided by a fastener-feeding module that can feed the fastener 114 supplied to the production line 200 in any continuous form, such as, for example, wound rolls or festoon containers. The fastener-feeding module can utilize any suitable hardware commonly used in converting operations for feeding continuous forms of materials.

While particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

What is claimed is:

1. A method for manufacturing a disposable fluid-handling article, the disposable fluid-handling article comprising at least two primary components made continuously from bulk starting materials, the method comprising the steps of:
a) feeding a first polymeric bulk starting material into at least one first polymeric extrusion apparatus disposed adjacent to a first collecting surface moving at a first velocity in relation to the first polymeric extrusion apparatus;
b) extruding a first molten stream of a first polymeric material from the first polymeric extrusion apparatus;
c) continuously forming a first primary component of the disposable fluid-handling article from the first molten stream;
d) feeding a second polymeric bulk starting material into at least one second polymeric extrusion apparatus disposed adjacent to a second collecting surface moving at a second velocity in relation to the second polymeric extrusion apparatus;
e) extruding a second molten stream of a second polymeric material from the second polymeric extrusion apparatus;
f) continuously forming a second primary component of the disposable fluid-handling article from the second molten stream;
g) joining the first and second primary components into a composite web, wherein the first primary component overlaps at least partially with the second primary component; and
h) severing the composite web in a direction generally perpendicular to a machine direction so as to form the disposable fluid-handling article.

2. The method of claim 1 further comprising the step of providing a third primary component of the disposable fluid-handling article for joining with at least one of the first and second primary components.

3. The method of claim 2 wherein the step of providing the third primary component comprises the steps of:
(i) feeding a third polymeric starting material into at least one third polymeric extrusion apparatus disposed adjacent to a third collecting surface moving at a third velocity in relation to the third polymeric extrusion apparatus;
(ii) extruding a third molten stream of a third polymeric material from the third polymeric extrusion apparatus; and
(iii) continuously forming a third component of the disposable fluid-handling article from the third molten stream.

4. The method of claim 1 further comprising the step of providing at least one auxiliary component of the disposable fluid-handling article for joining with at least one of the first and second primary components.

5. The method of claim 4 wherein the step of providing at least one auxiliary component comprises the steps of:
(i) feeding an auxiliary polymeric starting material into at least one auxiliary polymeric extrusion apparatus disposed adjacent to an auxiliary collecting surface moving at an auxiliary velocity in relation to the auxiliary polymeric extrusion apparatus;
(ii) extruding an auxiliary molten stream of an auxiliary polymeric material from the auxiliary polymeric extrusion apparatus; and
(iii) continuously forming an auxiliary component of the disposable fluid-handling article from the auxiliary molten stream.

6. The method of claim 1 wherein the first polymeric extrusion apparatus is a spun-bonded apparatus, a melt-blown apparatus, or a film-extruding apparatus, and wherein the second polymeric extrusion apparatus is a spun-bonded apparatus, a melt-blown apparatus, or a film forming apparatus.

7. The method of claim 3 wherein the third polymeric extrusion apparatus is a spun-bonded apparatus, a melt-blown apparatus, or a film-extruding apparatus.

8. The method of claim 5 wherein the auxiliary polymeric extrusion apparatus is a spun-bonded apparatus, a melt-blown apparatus, or a film-extruding apparatus.

9. The method of claim 1 wherein the first molten stream comprises continuous filaments.

10. The method of claim 9 wherein the first primary component is continuously formed from the continuous filaments by a process comprising the steps of:
(i) stretching the continuous filaments to reduce their cross-sectional diameter;
(ii) cooling the continuous filaments;
(iii) depositing the continuous filaments onto the first collecting surface; and
(iv) bonding the continuous filaments to form bonds between the continuous filaments.

11. The method of claim 1 wherein the first molten stream comprises melt-blown fibers.

12. The method of claim 11 wherein the first primary component is continuously formed from the melt-blown fibers by a process comprising the steps of:
(i) cooling the melt-blown fibers;
(ii) depositing the melt-blown fibers onto the first collecting surface; and
(iii) bonding the melt-blown fibers to form bonds between the melt-blown fibers.

13. The method of claim 1 wherein the first molten stream comprises a film.

14. The method of claim 1 wherein the disposable fluid-handling article is selected from a group consisting of a baby diaper, a baby training pants, an adult incontinence article, a feminine hygiene article, a baby pool diaper, a body wipe, a body wrap, a wound dressing, a dining bib, a pet grooming article, a cleaning article, and a polishing article.

15. The method of claim 1 wherein the first primary component is a topsheet, a backsheet, or a core of the disposable fluid-handling article.

16. The method of claim 1 wherein the second primary component is a topsheet, a backsheet, or a core of the disposable fluid-handling article.

17. The method of claim 3 wherein the third primary component is a topsheet, a backsheet, or a core of the disposable fluid-handling article.

18. The method of claim 5 wherein the auxiliary component is a leg cuff, a waist feature, or a fastener of the disposable fluid-handling article.

19. The method of claim 1 wherein the first velocity of the first collecting surface and the second velocity of the second collecting surface are substantially equal to each other.

20. The method of claim 1 wherein the first collecting surface is a screen, a perforated belt, a woven belt, a non-woven belt, a layer of spun-bonded filaments, a layer of melt-blown fibers, or a porous film, and wherein the second collecting surface is a screen, a perforated belt, a woven belt, a non-woven belt, a layer of spun-bonded filaments, a layer of melt-blown fibers, or a porous film.

21. A production line for producing a disposable fluid-handling article, the disposable fluid-handling article comprising at least two primary components made continuously from bulk starting materials, the production line comprising:
(i) a first primary component station for providing a first primary component, wherein the first primary component station includes at least one first extrusion module for forming the first primary component from one or more first polymeric bulk starting material by extrusion and formation of the first primary component continuously on the production line; and
(ii) a second primary component station adjacent to the first primary component station for providing a second primary component, wherein the second primary component station includes at least one second extrusion module for forming the second primary component from one or more second polymeric bulk starting material by extrusion and formation of the second primary component continuously on the production line.

22. The production line of claim 21 further comprising a third primary component station for providing a third primary component, wherein the third primary component station includes at least one third extrusion module for forming the third primary component from one or more third polymericic bulk starting material by extrusion and formation of the third primary component continuously on the production line.

23. The production line of claim 21 further comprising at least one auxiliary component station for providing an auxiliary component, wherein the auxiliary component station includes at least one auxiliary extrusion module for forming the at least one auxiliary component from one or more auxiliary polymericic bulk starting material by extrusion and formation of the auxiliary component continuously on the production line.

24. The production line of claim 21 wherein the first extrusion module is a spun-bonded module, a melt-blown module, or a film-forming module, and wherein a second extrusion module is a spun-bonded station, a melt-blown station, or a film-forming station.

25. The production line of claim 22 wherein the third extrusion module is a spun-bonded module, a melt-blown module, or a film-forming module.

26. The production line of claim 23 wherein the at least one auxiliary extrusion module is a spun-bonded module, a melt-blown module, or a film-forming module.

27. The production line of claim 25 wherein the spun-bonded module comprises:
(i) a cooling apparatus for cooling continuous filaments;
(ii) a drawing apparatus for stretching continuous filaments so as to reduce their cross-sectional diameter;
(iii) a collecting surface for depositing the continuous filaments; and
(iv) a bonding apparatus for bonding the continuous filaments so as to form bonds between the continuous filaments.

28. The production line of claim 25 wherein the melt-blown module comprises:
(i) a cooling apparatus for cooling melt-blown fibers;
(ii) a collecting surface for depositing the melt-blown fibers; and
(iii) a bonding apparatus for bonding the melt-blown fibers so as to form bonds between the melt-blown fibers.

29. The production line of claim 21 wherein the disposable fluid-handling article is selected from a group consisting of a baby diaper, a baby training pants, an adult incontinence article, a feminine hygiene article, a baby pool diaper, a body wipe, a body wrap, a wound dressing, a dining bib, a pet grooming article, a cleaning article, and a polishing article.

30. The production line of claim 21 wherein the first primary component is a topsheet, a backsheet, or a core of the disposable fluid-handling article.

31. The production line of claim 21 wherein the second primary component is a topsheet, a backsheet, or a core of the disposable fluid-handling article.

32. The production line of claim 22 wherein the third primary component is a topsheet, a backsheet, or a core of the disposable fluid-handling article.

33. The production line of claim 23 wherein the auxiliary component is a leg cuff, a waist feature, or a fastener of the disposable fluid-handling article.

34. The production line of claim 27 wherein the collecting surface is a screen, a perforated belt, a woven belt, a non-woven belt, a layer of spun-bonded filaments, a layer of melt-blown fibers, or a porous film.
